SEQUENCE LISTING (4 sequences)

SEQ ID NO: 1 - protein, 27 aa, Artificial Sequence
  Description of Artificial Sequence Synthetic peptide
FRKSKEKIGKEFKRIVQRIKDFLRNLV

SEQ ID NO: 2 - protein, 18 aa, Artificial Sequence
  Description of Artificial Sequence Synthetic peptide
KEFKRIVQRIKDFLRNLV

SEQ ID NO: 3 - protein, 9 aa, Artificial Sequence
  Description of Artificial Sequence Synthetic peptide
FRKSKEKIG

SEQ ID NO: 4 - protein, 170 aa, Human
MKTQRDGHSLGRWSLVLLLLGLVMPLAIIAQVLSYKEAVLRAIDGINQRSSDANLYRLLDLDPRPTMDGDPDTPKPVSFTVKETVCPRTTQQSPEDCDFKKDGLVKRCMGTVTLNQARGSFDISCDKDNKRFALLGDFFRKSKEKIGKEFKRIVQRIKDFLRNLVPRTES

(19) United States
(12) Patent Application Publication
(10) Pub. No.: US 2006/0057134 A1
(43) Pub. Date: Mar. 16, 2006

(54) Antibody against antibacterial peptide and utiliazation thereof

(76) Inventors: Teruo Kirikae, Tokyo (JP); Kiminori Toyooka, Kanagawa (JP); Yoshikazu Naiki, Los Angeles, CA (US); Hiroshi Tamura, Tokyo (JP); Akitoshi Ishizaka, Tokyo (JP); Satoru Hashimoto, Kyoto (JP)

Correspondence Address: SUGHRUE MION, PLLC, 2100 PENNSYLVANIA AVENUE, N.W., SUITE 800, WASHINGTON, DC, 20037, US

(21) Appl. No.: 10/522,153

(22) PCT Filed: Jul. 22, 2003
(86) PCT No.: PCT/JP03/09267

§ 371 Date: Oct. 31, 2005

(30) Foreign Application Priority Data
Jul. 22, 2002 (JP) 2002-213040
Mar. 14, 2003 (JP) 2003-70932

(51) Int. Cl. G01N 33/53; A61K 39/395; C07K 16/18

(52) U.S. Cl. 424/130.1; 435/7.1; 530/388.1

(57) ABSTRACT

An antibody which binds to a “peptide consisting of the amino acid sequence represented by SEQ ID NO:1”, and a method for assaying a “peptide comprising the amino acid sequence represented by SEQ ID NO:1” in a sample and a method for detecting a bacterial pneumonia, wherein the methods use the antibody or the like.

### TECHNICAL FIELD

The present invention relates to an antibody which binds to a peptide consisting of a specific amino acid sequence present in human-derived CAP18 (cationic antimicrobial protein of 18 kDa; antibacterial protein), as well as an assaying method for. CAP18 and the like and an assaying kit which use the same.

### BACKGROUND ART

JP-T-8-504085 (WO94/02589) discloses a full amino acid sequence containing a signal peptide of human-derived CAP18.

Minophagen Medical Review, vol. 43, No. 1, pp. 1-15 (1998) describes a full amino acid sequence of human-derived CAP18. Partial peptides consisting of 34, 32, 30, 27, 24 or 22 amino acid residues in the C-terminal region of human-derived CAP18 are also described.

Gendai-Iryo, vol. 28 (extra edition III), pp. 2367-2375 (1996) describes a full amino acid sequence of human-derived CAP18. Partial peptides consisting of 34, 30, 27, 24 or 22 amino acid residues in the C-terminal region of human-derived CAP18 are also described.

Shock; From Molecular and Cellular Level to Whole Body (Proceedings of the Third International Shock Congress-Shock '95, Hamamatsu, Japan, 21-23 October (1995)), and Okada, K., Ogata, H. eds. Elsevier Sciences B.V. pp. 109-115 (1996) describe a full amino acid sequence of human-derived CAP18. Partial peptides consisting of 34, 30, 27 or 24 amino acid residues in the C-terminal region of human-derived CAP18 is also described. Data of these peptides regarding their lipopolysaccharide (LPS) binding activity are also described.

Bacterial Endotoxins; Lipopolysaccharides From Genes to Therapy, Levin, J., Alving, C. R., Munford, R. S., Redl, H. eds. Wiley-Liss, Inc., New York, pp. 317-326 (1995) describes partial peptides consisting of 37 or 32 amino acid residues in the C-terminal region of human-derived CAP18.

However, there is neither description nor suggestion about an antibody which binds to a partial peptide of CAP18 (peptide consisting of the amino acid sequence represented by SEQ ID NO:1). Moreover, there is neither description nor suggestion about a method for assaying a “peptide comprising the amino acid sequence represented by SEQ ID NO:1” such as CAP 8 using this antibody, a kit therefor, and the like.

If an antibody which binds to a peptide characteristic of CAP18 is obtained, it can be utilized as a tool for the detection and assay of CAP18. Additionally, since the antibody is highly homogeneous and reproducible, and also can permanently be produced in a large amount, production costs can be remarkably reduced.

Also, if a method for assaying CAP18 or the like using the antibody or an assaying kit therefor is provided, it can be applied to a research reagent or a diagnostic agent for diseases relating to CAP18 and the like, and can also be possibly applied to monitoring of such diseases, etc.

### DISCLOSURE OF THE INVENTION

The present invention provides an antibody which binds to a “peptide consisting of the amino acid sequence represented by SEQ ID NO:1” (hereinafter referred to as “inventive antibody”).

The inventive antibody is preferably one which specifically binds to a “peptide consisting of the amino acid sequence represented by SEQ ID NO:2”, and this antibody is preferably a monoclonal antibody. Furthermore, preferably, the antibody belongs to the immunoglobulin subclass IgG1.

Also, the inventive antibody is preferably one which specifically binds to a “peptide consisting of the amino acid sequence represented by SEQ ID NO:3”, and the antibody is preferably a polyclonal antibody.

Also, the present invention provides a method for assaying a “peptide comprising the amino acid sequence represented by SEQ ID NO:1” in a sample, which comprises using an “inventive antibody” (hereinafter referred to as “inventive method”).

The inventive method is preferably carried out by steps comprising the following steps (a) and (b) (hereinafter referred to as “Inventive method-1”):
(a) bringing a solid phase into contact with a sample to thereby immobilize the “peptide comprising the amino acid sequence represented by SEQ ID NO:1” in the sample on the solid phase; and
(b) detecting the “peptide comprising the amino acid sequence represented by SEQ ID NO:1” immobilized on the solid phase in step (a) by using an inventive antibody.

The “inventive antibody” used in Inventive method-1 is preferably one which is labeled with a label or is capable of being labeled with a label. Furthermore, the detection of the “peptide comprising the amino acid sequence represented by SEQ ID NO:1” is preferably carried out by further using an “antibody which specifically binds to the inventive antibody and which is labeled with a label or is capable of being labeled with a label”.

Also, the inventive method is preferably carried out by steps comprising the following steps (a) and (b) (hereinafter referred to as “Inventive method-2”):
(a) bringing a “solid phase on which an inventive antibody (primary antibody) is immobilized”, a “sample” and an “inventive antibody (secondary antibody)” into contact to thereby form a sandwich complex of “the primary antibody immobilized on the solid phase—the peptide comprising the amino acid sequence represented by SEQ ID NO:1—the secondary antibody”; and
(b) detecting the sandwich complex formed in step (a).

Also, the method is more preferably carried out by steps comprising the following steps (a) to (c):
(a) bringing a sample into contact with a “solid phase on which an inventive antibody (primary antibody) is immobilized” to thereby form a complex of “the primary antibody immobilized on the solid phase—the peptide comprising the amino acid sequence represented by SEQ ID NO:1”;
(b) bringing an “inventive antibody (secondary antibody)” into contact with the solid phase to thereby form a sandwich complex of “the primary antibody immobilized on the solid phase—the peptide comprising the amino acid sequence represented by SEQ ID NO:1—the secondary antibody”; and
(c) detecting the sandwich complex formed in step (b).

The “secondary antibody” used in Inventive method-2 is preferably one which is labeled with a label or is capable of being labeled with a label. Furthermore, the detection of the complex in Inventive method-2 is preferably carried out by using an “antibody which specifically binds to the secondary antibody and which is labeled with a label or is capable of being labeled with a label”.

Also, the inventive method is preferably carried out by steps comprising the following steps (a) and (b) (hereinafter referred to as “Inventive method-3”):
(a) bringing a “solid phase on which a peptide consisting of the amino acid sequence represented by SEQ ID NO:1 is immobilized”, a “sample” and an “inventive antibody” into contact to thereby form a complex of “the peptide consisting of the amino acid sequence represented by SEQ ID NO:1 immobilized on the solid phase—the inventive antibody” and a complex of “the peptide comprising the amino acid sequence represented by SEQ ID NO:1 in the sample—the inventive antibody”; and
(b) detecting at least one of the complex of “the peptide consisting of the amino acid sequence represented by SEQ ID NO:1 immobilized on the solid phase—the inventive antibody” and the complex of “the peptide comprising the amino acid sequence represented by SEQ ID NO:1 in the sample—the inventive antibody”.

This method is more preferably carried out by steps comprising the following steps (a) to (c):
(a) bringing a sample into contact with an “inventive antibody” to thereby form a complex-A of “the peptide comprising the amino acid sequence represented by SEQ ID NO:1—the inventive antibody”;
(b) bringing a “mixture comprising the complex-A and the inventive antibody which does not form the complex-A” obtained in step (a) into contact with a “solid phase on which a peptide consisting of the amino acid sequence represented by SEQ ID NO:1 is immobilized” to thereby form a complex of “the peptide consisting of the amino acid sequence represented by SEQ ID NO:1 immobilized on the solid phase—the inventive antibody”; and
(c) detecting the complex formed in step (b).

The “inventive antibody” used in Inventive method-3 is preferably one which is labeled with a label or is capable of being labeled with a label. It is also preferable to carry out the detection of the complex in Inventive method-3 by using an “antibody which specifically binds to the inventive antibody and which is labeled with a label or is capable of being labeled with a label”.

The “sample” used in the inventive methods is preferably a body fluid.

Also, the present invention provides a kit for assaying a “peptide comprising the amino acid sequence represented by SEQ ID NO:1”, which comprises the following components (A) and (B) (hereinafter referred to as “Inventive kit-1”):
(A) a solid phase; and
(B) an inventive antibody.

The “inventive antibody” used in Inventive kit-1 is preferably one which is labeled with a label or is capable of being labeled with a label.

Also, the present invention provides a kit for assaying a “peptide comprising the amino acid sequence represented by SEQ ID NO:1”, which comprises the following components (A) and (B) (hereinafter referred to as “Inventive kit-2”):
(A) a solid phase on which an inventive antibody (primary antibody) is immobilized; and
(B) an inventive antibody (secondary antibody).

The “inventive antibody” used in Inventive kit-2 is preferably one which is labeled with a label or is capable of being labeled with a label.

Also, the present invention provides a kit for assaying a “peptide comprising the amino acid sequence represented by SEQ ID NO:1”, which comprises the following components (A), (B) and (C) (hereinafter referred to as “Inventive kit-3”):
(A) a solid phase on which a peptide consisting of the amino acid sequence represented by SEQ ID NO:1 is immobilized;
(B) an inventive antibody; and
(C) an antibody which specifically binds to the inventive antibody and which is labeled with a label or is capable of being labeled with a label.

Also, the present invention provides a method for detecting a bacterial pneumonia, which comprises assaying an antigen in a sample which can be detected by an “inventive antibody” or an “antibody capable of specifically binding to CAP18” to thereby detect a bacterial pneumonia in a patient from which the sample is obtained (hereinafter referred to as “inventive detection method”).

In the inventive detection method, the antigen in the sample is preferably an antigen selected from the group consisting of a “peptide comprising the amino acid sequence represented by SEQ ID NO:1”, a “peptide comprising the amino acid sequence represented by SEQ ID NO:2”, a “peptide comprising the amino acid sequence represented by SEQ ID NO:3” and CAP18.

In the inventive detection method, preferably, the assay is immunologically carried out by using an antibody selected from the group consisting of an “antibody capable of binding to a peptide consisting of the amino acid sequence represented by SEQ ID NO:1”, an “antibody capable of specifically binding to a peptide consisting of the amino acid sequence represented by SEQ ID NO:2”, an “antibody capable of specifically binding to a peptide consisting of the amino acid sequence represented by SEQ ID NO:3” and an “antibody capable of specifically binding to CAP18”.

In the inventive detection method, the detection of a bacterial pneumonia is preferably carried out by evaluating or monitoring the presence or absence of infection, degree or type of the bacterial pneumonia.

Also, in the inventive detection method, the assay is preferably carried out by the above inventive method.

The present invention provides a kit for diagnosing a bacterial pneumonia, which comprises an “inventive antibody” (hereinafter referred to as “inventive diagnostic kit”).

The inventive diagnostic kit is preferably a kit consisting of any one of Inventive kits 1 to 3.

The present invention provides a diagnostic agent which comprises, as an active ingredient, an “inventive antibody” (hereinafter referred to as “inventive diagnostic agent”).

### BRIEF DESCRIPTION OF THE DRAWINGS

FIG. 1 is photographs showing immunological staining images of cells using a monoclonal antibody Toyo6E3 (morphology of an organism).

FIG. 2 shows detection and determination results of a peptide by direct ELISA.

FIG. 3 shows detection and determination results of CAP18 by indirect ELISA (sandwich method).

FIG. 4 is a photograph showing the results of a detection of CAP18 in cell extracts by Western blotting.

FIG. 5 is a photograph showing detection results of serum CAP18 by immunoprecipitation.

FIG. 6 shows schematic views (A and B) and a photograph (C) of detection and determination results of release of CAP18 from human neutrophiles stimulated with fMLP.

### BEST MODE FOR CARRYING OUT THE INVENTION

The present inventors made an effort to solve the above problems and could provide an antibody which binds to a peptide consisting of the amino acid sequence represented by SEQ ID NO:1. Also, the present inventors could provide a method for assaying a “peptide comprising the amino acid sequence represented by SEQ ID NO:1” such as CAP18 and an assaying kit therefor, which use the antibody. Thus, the present invention has been completed.

Embodiments of the present invention are described below.

<1> Inventive Antibody

A full amino acid sequence comprising a human-derived CAP18 signal peptide part is represented by SEQ ID NO:4. The signal peptide part corresponds to the −30th to −1st amino acids in SEQ ID NO:4.

The inventive antibody is an antibody which binds to a “peptide consisting of the amino acid sequence represented by SEQ ID NO:1”. The amino acid sequence represented by SEQ ID NO:1 corresponds to the 109th to 135th amino acids in SEQ ID NO:4.

The inventive antibody can be obtained by the usual antibody production method using, as an antigen, a peptide consisting of the amino acid sequence represented by SEQ ID NO:1 (peptide of SEQ ID NO:1 itself) or a partial peptide thereof (hereinafter sometimes referred to as “antigen peptide”).

The partial peptide includes a peptide consisting of the amino acid sequence represented by SEQ ID NO:2 and a peptide consisting of the amino acid sequence represented by SEQ ID NO:3. The amino acid sequence represented by SEQ ID NO:2 corresponds to the 118th to 135th amino acids in SEQ ID NO:4, and the amino acid sequence represented by SEQ ID NO:3 corresponds to the 109th to 117th amino acids in SEQ ID NO:4.

The antigen peptide can be produced based on its sequence by a known method for chemically synthesizing a peptide (for example, a liquid phase synthesis, solid phase synthesis, etc.; see Nobuo Izumiya, Tetsuo Kato, Tohiko Aoyagi, Michinori Waki, Fundamentals and Experiments of Peptide Syntheses, 1985, Maruzen).

Also, the antigen peptide can be produced by producing a polynucleotide (DNA or RNA) corresponding to the amino acid sequence of the antigen peptide and applying gene engineering techniques using the polynucleotide.

In this connection, a “peptide comprising the amino acid sequence represented by SEQ ID NO:1” such as CAP18 can be used as an antigen peptide, so long as an antibody which binds to a “peptide consisting of the amino acid sequence represented by SEQ ID NO:1” can be obtained. In such a case, since an antibody which binds to a peptide other than the “peptide consisting of the amino acid sequence represented by SEQ ID NO:1” may also be obtained, a “peptide consisting of the amino acid sequence represented by SEQ ID NO:1” is used to select an antibody binding thereto.

An antigen peptide thus produced can be purified by a method for isolating and purifying a peptide which is well known in the art of the protein chemistry. Examples include extraction, recrystallization, salting out using ammonium sulfate, sodium sulfate, etc., centrifugation, dialysis, ultrafiltration, absorption chromatography, ion exchange chromatography, hydrophobic chromatography, normal-phase chromatography, reverse-phase chromatography, gel filtration, gel permeation chromatography, affinity chromatography, electrophoresis, countercurrent distribution, and the like, and combinations thereof.

The amino acid sequence of the antigen peptide produced is determined by a known amino acid sequencing method (for example, Edman degradation method, etc.) to confirm that the antigen peptide has been correctly produced.

When a relatively low molecular peptide such as a peptide consisting of an amino acid sequence of SEQ ID NO:1, SEQ ID NO:2 or SEQ ID NO:3 is used as an antigen, the antigen is preferably bound to a carrier such as hemocyanin, ovalbumin or γ-globulin.

The inventive antibody may be a monoclonal antibody or a polyclonal antibody. The production of the inventive antibody can be accomplished as follows depending on the type of the antibody desired, i.e., a monoclonal antibody or a polyclonal antibody.

A monoclonal inventive antibody can be produced by using an antigen peptide described above by a method of Kohler and Milstein (Nature, 256, 495-497 (1975)).

For example, an antigen peptide is administered intraperitoneally, subcutaneously or to a footpad of an animal to be immunized such as a mouse, a rat, a guinea pig, a rabbit, a goat, a sheep, a horse, a pig, a dog, a cat or a chicken. Among these animals to be immunized, a mouse is preferably used. Thus, the inventive antibody is preferably a mouse-derived antibody.

From an animal to be immunized, a spleen cell, a lymphocyte, peripheral blood or the like is collected and is subjected to cell fusion with a myeloma cell which is a tumor cell line to prepare a hybridoma. Although the myeloma cell used in the cell fusion may be a cell line from any of various mammalian animals, it is preferred to use a cell line of an animal homologous to the immunized animal. Furthermore, in order to distinguish the non-fused cell and the fused cell, the myeloma cell having a marker is preferably used so as to proliferate only a hybridoma without survival of the non-fused myeloma cell. Moreover, since an antibody of interest is easily obtained from the culture supernatant of the hybridoma, a myeloma cell line which does not secret a specific immunoglobulin is preferably used.

The resulting hybridoma is continuously proliferated, and a hybridoma cell line which continuously produces an antibody capable of specifically binding to an antigen is selected.

The hybridoma cell line thus selected is cultured in a suitable medium to obtain a monoclonal antibody in the culture. It is also possible to produce a monoclonal antibody in a large amount by culturing the hybridoma cell line described above in vivo, for example, in the abdominal cavity in a mouse, followed by isolation from the ascites. The monoclonal antibody thus obtained may be purified by the usual antibody purification method.

A polyclonal inventive antibody can be produced as described below by using the above-described antigen peptide.

Similarly to the above-described production method of the monoclonal antibody, an antigen peptide is administered to an animal to be immunized. Herein, a rabbit is preferably used as the animal to be immunized.

When the animal to be immunized is immunized, an adjuvant is preferably used in combination since an antibody-producing cell is activated. When booster immunization is carried out 2 to 3 weeks after the first immunization serves by the usual method, antiserum having a high titer can be produced. About 1 week after the final immunization, blood is collected and serum is separated. The serum is heated to inactivate a complement, and then an immunoglobulin fraction can be purified by the usual antibody purification method.

The antibody purification method includes salting out using sodium sulfate, ammonium sulfate, etc., low temperature alcohol precipitation, selective precipitation separation using polyethylene glycol or an isoelectric point, electrophoresis, ion exchange chromatography by using an ion exchanger such as DEAE (diethylaminoethyl)-derivative or CM (carboxymethyl)-derivative, affinity chromatography using protein A or protein G, hydroxyapatite chromatography, antigen-immobilizing immunosorbent chromatography, gel filtration, supercentrifugation, and the like.

The inventive antibody can be treated with a protease which does not decompose an antigen binding site (Fab) (for example, plasmin, pepsin, papain, etc.) to form an Fab-containing fragment. The Fab-containing fragment of the antigen includes Fabc, (Fab′)₂ and the like, in addition to Fab. They are included in the concept of the “inventive antibody” in the present specification.

Also, when the nucleotide sequence of a gene encoding the inventive antibody or the amino acid sequence of the inventive antibody is determined, a fragment containing Fab of the inventive antibody or a chimera antibody (for example, a chimera antibody containing Fab of the inventive antibody, etc.) by genetic engineering techniques. The fragment or chimera antibody containing Fab of the inventive antibody may also be included in the concept of the “inventive antibody” in the present specification, so long as it binds to an antigen peptide.

Whether or not the produced antibody binds to the antigen peptide, or whether or not the antibody specifically binds to the antigen peptide can easily be determined by those skilled in the art by the usual method using other substance which can be used as an antigen peptide or an antigen (for example, peptide of other kind) or the like.

Preferably, the inventive antibody belongs to the immunoglobulin subclass IgG1. The antibody belonging to the immunoglobulin subclass IgG1 can be obtained, for example, by a screening method using an anti-IgG1 antibody or the like.

Also, other preferred embodiment of the inventive antibody is an antibody which specifically binds to a “peptide consisting of the amino acid sequence represented by SEQ ID NO:3”. This antibody is preferably a polyclonal antibody.

The inventive antibody may be one which is labeled with a label or is capable of being labeled with a label. Although the label which can be used for labeling is not particularly limited, so long as it can ordinarily be used for labeling a protein. Examples include an enzyme (e.g., peroxidase, alkaline phosphatase, β-galactosidase, luciferase, acetylcholine esterase, glucose oxidase, etc.), a radioisotope (¹²⁵I, ¹³¹I, ³H, etc.), a fluorescent dye (Alexa Fluor® 488, fluorescein isothiocyanate (FITC), 7-amino-4-methylcoumarine-3-acetic acid (AMCA), dichlorotriazinyl aminofluorescein (DTAF), tetramethyl Rhodamine isothiocyanate (TRITC), Lussamine rhodamine B, Texas red, phycoerythrin (PE), umbeliferon, europium, phycocyanin, Tricolor, cyanin, etc.), a chemiluminescent substance (luminol, etc.), hapten (dinitrofluorobenzene, adenosine monophosphate (AMP), 2,4-dinitroaniline, etc.), either member of a specific binding pair (biotin and avidins (streptoavidin, etc.), lectin and a saccharide chain, an agonist and an agonist receptor, heparin and antithrombin III (ATIII), a polysaccharide and its binding protein (hyaluronic acid and a hyaluronic acid-binding protein (HABP), etc.)), and the like.

A method for labeling the inventive antibody with a label can be appropriately selected from known methods suitable for the label, for example, in the case of labeling the inventive antibody with an enzyme, from a glutaraldehyde method, a periodic acid crosslinking method, a maleimide crosslinking method, a carbodiimide method, an activated ester method, and the like, and in the case of labeling the inventive antibody with a radioisotpoe, from a chloramine T method, a lactoperoxidase method, and the like (see, Sequel of Biochemistry Experiments, 2, “Protein Chemistry (2nd vol.)”, Tokyo Kagaku Dojin (1987)). For example, in the case of using biotin as a label, a method using an N-hydroxysuccinimide ester derivative of biotin or a hydrazide derivative can be used (see, Avidin-Biotin Chemistry: A Handbook, p. 57-63, Pierce Chemical Company, 1994).

Also, when the inventive antibody is stored, distributed or used, other components can be added, so long as the functions and effects of the inventive antibody are not substantially damaged. For example, it can contain an excipient, a buffering agent, a stabilizer, a preservative and the like which are used in preparing a usual reagent. Examples include phosphate buffered saline (PBS), sodium azide (NaN₃), bovine serum albumin (BSA), and the like.

<2> Inventive Method

The inventive method is a method for assaying a “peptide comprising the amino acid sequence represented by SEQ ID NO:1” in a sample, which comprises using an “inventive antibody”.

The explanation of the “inventive antibody” used in the inventive method is similar to that of the “inventive antibody” described above. The inventive antibody is preferably one which is labeled with a label or is capable of being labeled with a label in order to facilitate the detection, The label used for labeling and the labeled method are similar to those described above.

Also, the “sample” is not particularly limited, and includes one containing a “peptide comprising the amino acid sequence represented by SEQ ID NO:1” such as CAP18 and one which is capable of containing it. Examples include a standard solution of CAP18, a cell or tissue extract, a cell culture supernatant, a body fluid and the like, and a body fluid is preferred. The “body fluid” includes, for example, blood (used as a concept including serum and plasma in the present specification), urine, saliva, sweat, tear, an intraarticular fluid, and the like.

Also, the “peptide comprising the amino acid sequence represented by SEQ ID NO:1” which is assayed is not particularly limited, so long as it contains at least the amino acid sequence represented by SEQ ID NO:1, and it is a concept including polypeptides. Examples include CAP18, a partial peptide thereof (comprising the amino acid sequence represented by SEQ ID NO:1), a peptide itself consisting of the amino acid sequence represented by SEQ ID NO:1, and the like.

A method for “assaying” a “peptide comprising the amino acid sequence represented by SEQ ID NO:1” in a sample is not particularly limited, so long as it is a method capable of detecting the peptide by using the inventive antibody. In this connection, the “assaying” in the inventive method is a concept including not only a quantitative detection but also a qualitative detection (detection of the presence or absence). Accordingly, the “assaying” in the present invention includes screening of the peptide in a sample.

A method for assaying a “peptide comprising the amino acid sequence represented by SEQ ID NO:1” by using the inventive antibody includes those described below.
i) A method in which a “peptide comprising the amino acid sequence represented by SEQ ID NO:1” in a sample is immobilized on a solid phase and detected directly (so-called direct ELISA).
ii) A method in which a sample is brought into contact with a solid on which an inventive antibody (primary antibody) is immobilized and then with an inventive antibody (secondary antibody) to thereby form a sandwich complex, and then the complex is detected (so-called sandwich method).
iii) A method in which a “peptide consisting of the amino acid sequence represented by SEQ ID NO:1” immobilized on a solid phase, a sample and an inventive antibody (the sample and the inventive antibody may be brought into contact in advance) are allowed to coexist while allowing the “peptide consisting of the amino acid sequence represented by SEQ ID NO:1” immobilized on the solid phase and a “peptide comprising the amino acid sequence represented by SEQ ID NO:1” in the sample to compete with each other, and the inventive antibody bound to the solid phase is detected to thereby assay the “peptide comprising the amino acid sequence represented by SEQ ID NO:1” in the sample (so-called inhibition method).
iv) A method in which a sample is brought into contact with microparticles on which an inventive antibody is immobilized and then with an inventive antibody to thereby aggregate the microparticles, and then the aggregate (or precipitate) is detected (so-called aggregation method).
   <2>-1 Inventive Method-1

The inventive method is preferably carried out by direct ELISA. Specifically, the inventive method is preferably carried out by steps comprising the following steps (a) and (b) (Inventive method-1):
(a) bringing a sample into contact with a solid phase to thereby immobilize a “peptide comprising the amino acid sequence represented by SEQ ID NO:1” in the sample on the solid phase;
(b) detecting the “peptide comprising the amino acid sequence represented by SEQ ID NO:1” immobilized on the solid phase in step (a) by using the inventive antibody.

Each step is discussed in detail below.

Step (a):

Step (a) is a step for bringing a solid phase into contact with a sample to thereby immobilize the “peptide comprising the amino acid sequence represented by SEQ ID NO:1” in the sample on the solid phase. The “solid phase” on which the “peptide comprising the amino acid sequence represented by SEQ ID NO:1” in the sample is immobilized is not particularly limited, so long as it is capable of immobilizing the peptide and is insoluble in water, a sample or an assaying reaction solution. The shape of the solid phase includes a plate (e.g., well of microplate, etc.), a tube, a bead, membrane, gel, a microparticulate solid carrier (gelatin particle, kaolin particle, synthetic polymer particle such as latex, etc.) and the like. Among these, a microplate is preferred in view of accurate determination and convenient handling.

The material of the solid phase includes polystyrene, polypropylene, polyvinyl chloride, nitrocellulose, nylon, polyacrylamide, Teflon®, polyaromer, polyethylene, glass, agarose, and the like. Among these, a plate made of polystyrene is preferred.

As the method for immobilizing the “peptide comprising the amino acid sequence represented by SEQ ID NO:1” in the sample to the solid phase, a usual method for preparing an immobilized enzyme such as a physical adsorption method, a covalent bond method and an inclusion method can be used (see, Immobilized Enzymes, 1975, Kodansha, pp. 9 to 75).

Among these, a physical adsorption method is preferred in view of its simple operation and wide use.

A specific method for physical adsorption is described below.

A sample is diluted in a buffer at pH 7 to 10 (for example, carbonate buffer, phosphate buffer, PBS, etc.) and added to a solid phase (for example, microplate), followed by storing at about 20 to 37° C. for 1 to 2 hours or at 4° C. overnight for immobilization.

The explanations of the “sample”” used herein and the “peptide comprising the amino acid sequence represented by SEQ ID NO:1” to be assayed are similar to those described above.

The method for bringing a solid phase into contact with a sample is not particularly limited, so long as the solid phase is brought into contact with the “peptide comprising the amino acid sequence represented by SEQ ID NO:1” in the sample. For example, the contact can be carried out by adding the sample to the solid phase, the contact can be carried out by adding the solid phase to the sample, or both can be added simultaneously to a separate container. The method of the contact is not limited thereto, and may be appropriately determined by those skilled in the art based on the shape and material of the solid phase, and the like.

After bringing the solid phase into contact with the sample, the solid phase and the liquid phase are separated. It is preferred to wash the surface of the solid phase with a washing solution, if necessary, to remove any non-specifically adsorbed substances or non-reacted components in the sample.

The washing solution is preferably a buffer (for example, phosphate buffer, PBS, Tris-HCl buffer, etc.) to which a nonionic surfactant such as a Tween-based surfactant is added.

The solid phase is brought into contact with a sample to thereby immobilize the “peptide comprising the amino acid sequence represented by SEQ ID NO:1” in the sample on the solid phase.

Step (b):

Step (b) is a step for detecting the “peptide comprising the amino acid sequence represented by SEQ ID NO:1” immobilized on the solid phase in step (a) by using the inventive antibody. The explanation of the inventive antibody is similar to that described above.

The method for detecting the “peptide comprising the amino acid sequence represented by SEQ ID NO:1” immobilized on the solid phase is not particularly limited, so long as the detection is carried out by using the inventive antibody. For example, when the inventive antibody is labeled with a label, the inventive antibody is bound to the “peptide comprising the amino acid sequence represented by SEQ ID NO:1” immobilized on the solid phase, and the label is then detected to thereby detect the peptide.

The method for detecting the label may be appropriately selected from known detection means depending on the type of the label. For example, in the case where one member of a specific binding pair (for example, biotin) is used as the label, an enzyme (for example, peroxidase, etc.) bound to the other member which specifically binds thereto (for example, streptoavidin) is added to thereby form a specific binding pair. Then, a substrate for the enzyme (for example, hydrogen peroxide (in the case where the enzyme is peroxidase)) and a chromogenic substance (for example, 3,3′,5,5′-tetramethylbenzidine (TMB), diaminobenzidine, etc.) are added and the degree of the color development by a product of the enzyme reaction is assayed based on an absorbance to thereby detect the label.

Also, when a radioisotope, a fluorescent dye or a chemiluminescent substance is used as a label, the method includes methods assaying a radioactivity count, fluorescent intensity, fluorescent polarization, luminescent intensity and the like.

By such detection of the label, the “peptide comprising the amino acid sequence represented by SEQ ID NO:1” immobilized on the solid phase can be detected to thereby assay the “peptide comprising the amino acid sequence represented by SEQ ID NO:1” in the sample. Since this method is direct ELISA, detection of the label in a large amount means that the “peptide comprising the amino acid sequence represented by SEQ ID NO:1” immobilized on the solid phase is present in a large amount, i.e., the “peptide comprising the amino acid sequence represented by SEQ ID NO:1” is present in a large amount in the sample.

When a qualitative assay (detection of the presence or absence) of the “peptide comprising the amino acid sequence represented by SEQ ID NO:1” is desired, the presence or absence of the detection of the label can be used, as it is, as an assaying result.

Alternatively, when a quantitative assay (assay of the concentration, etc.) of the “peptide comprising the amino acid sequence represented by SEQ ID NO:1” is desired, the absorbance, radioactivity count, fluorescent intensity, luminescent intensity and the like can be used, as they are, as an index of the quantity of the “peptide comprising the amino acid sequence represented by SEQ ID NO:1”. Furthermore, a calibration curve or equation for the relationship between the “peptide comprising the amino acid sequence represented by SEQ ID NO:1” and the detection results (for example, absorbance) of the standard substance is prepared in advance by using a standard solution of the “peptide comprising the amino acid sequence represented by SEQ ID NO:1” having a known concentration, and the concentration of the “peptide comprising the amino acid sequence represented by SEQ ID NO:1” in the sample can be obtained based on it. Also, when urine is used as a sample, the resultant concentration can be corrected on the basis of the concentration of other component in the urine, such as creatinine.

Moreover, the detection of the “peptide comprising the amino acid sequence represented by SEQ ID NO:1” immobilized on the solid phase is preferably carried out by using an “antibody which specifically binds to the inventive antibody and which is labeled with a label or is capable of being labeled with a label”.

The “antibody which specifically binds to the inventive antibody” is not particularly limited, so long as it specifically binds to the inventive antibody. For example, depending on an animal from which the inventive antibody is derived or an immunoglobulin class, an antibody which specifically binds to the immunoglobulin class of the animal is exemplified. For example, when the inventive antibody is an immunoglobulin (mouse-derived IgG1), the anti-mouse IgG1 antibody can be used as an “antibody which specifically binds to the inventive antibody”. The label which can be used for labeling the “antibody which specifically binds to the inventive antibody”, the labeling method, the label detecting method and the like are similar to those described in the above.

<2>-2 Inventive Method-2

The inventive method is also preferably carried out by a sandwich method. Specifically, the inventive method is also preferably carried out by steps comprising the following steps (a) and (b) (Inventive method-2):
(a) bringing a “solid phase on which an inventive antibody (primary antibody) is immobilized”, a “sample” and an “inventive antibody (secondary antibody)” into contact to thereby form a sandwich complex of “the primary antibody immobilized on the solid phase—the peptide comprising the amino acid sequence represented by SEQ ID NO:1—the secondary antibody”;
(b) detecting the sandwich complex formed in step (a).

In step (a), the three members, i.e., the “solid phase on which an inventive antibody (primary antibody) is immobilized”, the “sample” and the “inventive antibody (secondary antibody)” can be brought into contact simultaneously, or, alternatively, the former two are first brought into contact and then into contact with the latter one, or the latter two are first brought into contact and then into contact with the former one. Among these, preferably, the former two are first brought into contact and then into contact with the latter one. That is, the method is more preferably carried out by steps comprising the following steps (a) to (c):
(a) bringing a sample into contact with a “solid phase on which an inventive antibody (primary antibody) is immobilized” to thereby form a complex of “the primary antibody immobilized on the solid phase—the peptide comprising the amino acid sequence represented by SEQ ID NO:1”;
(b) bringing an “inventive antibody (secondary antibody)” into contact with the solid phase described above to thereby form a sandwich complex of “the primary antibody immobilized on the solid phase—the peptide comprising the amino acid sequence represented by SEQ ID NO:1—the secondary antibody”; and
(c) detecting the sandwich complex formed in step (b).

Each step is discussed in detail below.

Step (a):

Step (a) is a step for bringing a sample into contact with a “solid phase on which an inventive antibody (primary antibody) is immobilized” to thereby form a complex of “the primary antibody immobilized on the solid phase—the peptide comprising the amino acid sequence represented by SEQ ID NO:1”.

The explanation of the “inventive antibody (primary antibody)” is similar to that described above.

Also, the explanation of the “solid phase” on which the inventive antibody (primary antibody) is immobilized is similar to that in Inventive method-1. Furthermore, the method for immobilizing the inventive antibody (primary antibody) on the solid phase is similar to that in Inventive method-1, and a usual method such as a physical adsorption method, a covalent bond method or an inclusion method can be used. Among these, a physical adsorption method is preferred because of its simple operation and wide use.

A specific method for physical adsorption is described below.

An inventive antibody (primary antibody) is diluted in a buffer at pH 7 to 9 (for example, Tris buffer, phosphate buffer, PBS, carbonate buffer, etc.) and added to a solid phase (for example, microplate), followed by storing at about 20 to 37° C. for 1 to 2 hours or at 4° C. overnight to thereby immobilize the inventive antibody (primary antibody) on the solid phase.

Also, a part of the surface on which the inventive antibody (primary antibody) is not immobilized may remain on the surface of the solid phase on which the inventive antibody (primary antibody) is immobilized. When the “peptide comprising the amino acid sequence represented by SEQ ID NO:1” present in the sample is non-specifically immobilized thereon, correct assaying results might not be obtained. Accordingly, it is preferred that prior to the contact of the sample with the solid phase, a blocking agent is added to cover the part where the inventive antibody (primary antibody) is not immobilized. The blocking agent includes serum, serum albumin, casein, skimmed milk, gelatin, pluronic and the like, and those which are commercially available as blocking agents can be used.

Specifically, the method for blocking includes a method in which a blocking agent is added, followed by storing at about 37° C. for 30 minutes to 2 hour or at room temperature (15 to 25° C.) for 1 to 2 hours.

The explanation of the “sample” used herein is similar to that described above.

The method for bringing the solid phase into contact with a sample is not particularly limited, so long as the inventive antibody (primary antibody) immobilized on the solid phase is brought into contact with the “peptide comprising the amino acid sequence represented by SEQ ID NO:1” in the sample. Other explanations of the “method for bringing a solid phase into contact with a sample” are similar to those in Inventive method-1.

After both are brought into contact, the reaction is preferably carried out at 4 to 37° C., preferably 20 to 37° C., for about 1 to 4 hours in order to sufficiently bind the inventive antibody (primary antibody) and the “peptide comprising the amino acid sequence represented by SEQ ID NO:1” in the sample.

After the reaction, the solid phase and the liquid phase are separated. It is preferred to wash the surface of the solid phase with a washing solution, if necessary, in order to remove any non-specifically adsorbed substances or non-reacted components in the sample. The explanation of the washing solution used herein is similar to that in Inventive method-1.

A complex of “the inventive antibody immobilized on the solid phase (primary antibody)—the peptide comprising the amino acid sequence represented by SEQ ID NO:1” is formed by bringing the sample into contact with the solid phase on which the inventive antibody (primary antibody) is immobilized.

Step (b):

Step (b) is a step for bringing an “inventive antibody (secondary antibody)” into contact with the solid phase after step (a) to thereby form a sandwich complex of “the primary antibody immobilized on the solid phase—the peptide comprising the amino acid sequence represented by SEQ ID NO:1—the secondary antibody”.

The explanation of the “inventive antibody (secondary antibody)” is similar to that described above.

The secondary antibody is preferably labeled with a label or is capable of being labeled with a label in order to facilitate the detection. The label used in labeling and the method for labeling are similar to those described above.

The contact between the solid phase described above after step (a) and the inventive antibody (secondary antibody) can be carried out similarly to the “method for bringing a solid phase into contact with a sample” described above. Similarly to the above, the solid phase and the liquid phase are separated after the reaction and it is preferred that the surface of the solid phase is washed with a washing solution, if necessary, in order to remove any non-specifically adsorbed substances or non-reacted components in the sample. The washing solution which can be used is also similar to that described above.

The secondary antibody is brought into contact with the above-described solid phase after step (a) (wherein a complex of “the primary antibody immobilized on the solid phase—the peptide comprising the amino acid sequence represented by SEQ ID NO:1” is formed) to thereby form a sandwich complex of “the primary antibody immobilized on the solid phase—the peptide comprising the amino acid sequence represented by SEQ ID NO:1—the secondary antibody” is formed.

Step (c):

Step (c) is a step for detecting the sandwich complex formed in step (b).

The method for detecting the sandwich complex is not particularly limited. For example, when the secondary antibody is labeled with a label, the label is detected to thereby detect the complex.

The method for detecting the label may be appropriately selected from known detection means depending on the type of the label. Other explanations are similar to those in Inventive method-1.

By the detection of the label, the sandwich complex can be detected to thereby assay the “peptide comprising the amino acid sequence represented by SEQ ID NO:1” in the sample. Since this method is a sandwich method, detection of the label in a large amount means that the sandwich complex is present in a large amount, i.e., the “peptide comprising the amino acid sequence represented by SEQ ID NO:1” is present in a large amount in the sample.

The explanations of a qualitative assay (detection of the presence or absence) and a quantitative assay (e.g., assay of the concentration, etc.) of the “peptide comprising the amino acid sequence represented by SEQ ID NO:1” are similar to those in Inventive method-1.

Furthermore, the detection of the complex in Inventive method-2 is preferably carried out by using an “antibody which specifically binds to the secondary antibody and which is labeled with a label or is capable of being labeled with a label”.

The explanation of the “antibody which specifically binds to the secondary antibody (inventive antibody)” is also similar to that of the “antibody which specifically binds to the inventive antibody” in Inventive method-1.

<2>-3 Inventive Method-3

The inventive method is also preferably carried out by an inhibition method. Specifically, the inventive method is also preferably carried out by steps comprising the following steps (a) and (b) (Inventive method-3):
(a) bringing a “solid phase on which a peptide consisting of the amino acid sequence represented by SEQ ID NO:1 is immobilized”, a “sample” and an “inventive antibody” into contact to thereby form a complex of “the peptide consisting of the amino acid sequence represented by SEQ ID NO:1 immobilized on the solid phase—the inventive antibody” and a complex of “the peptide comprising the amino acid sequence represented by SEQ ID NO:1 in the sample—the inventive antibody”;
(b) detecting at least one of the complex of “the peptide consisting of the amino acid sequence represented by SEQ ID NO:1 immobilized on the solid phase—the inventive antibody” and the complex of “the peptide comprising the amino acid sequence represented by SEQ ID NO:1 in the sample—the inventive antibody”.

In step (a), the three members, namely, the “inventive antibody”, the “sample” and the “solid phase on which a peptide consisting of the amino acid sequence represented by SEQ ID NO:1 is immobilized” may be brought into contact simultaneously, or, alternatively, the former two are first brought into contact and then into contact with the latter one, or the latter two are first brought into contact and then into contact with the former one. It is preferred that the former two are first brought into contact and then into contact with the latter one.

Also, in step (b), among the complex of the “peptide consisting of the amino acid sequence represented by SEQ ID NO:1 immobilized on the solid phase—the inventive antibody” and the complex of the “peptide comprising the amino acid sequence represented by SEQ ID NO:1 in the sample—the inventive antibody”, only the former can be detected, or only the latter can be detected, or both can be detected. In this inventive method, it is preferred to detect only the former. That is, the inventive method is preferably carried out by steps comprising the following steps (a) to (c):
(a) bringing an “inventive antibody” into contact with a sample to thereby form a complex-A of “the peptide comprising the amino acid sequence represented by SEQ ID NO:1—the inventive antibody”;
(b) bringing a “mixture comprising the complex-A and the inventive antibody which does not form the complex-A” obtained in step (a) into contact with the “solid phase on which a peptide consisting of the amino acid sequence represented by SEQ ID NO:1 is immobilized” to thereby form a complex of “the peptide consisting of the amino acid sequence represented by SEQ ID NO:1 immobilized on the solid phase—the inventive antibody”; and
(c) detecting the complex formed in step (b).

Each step is described below in detail.

Step (a):

Step (a) is a step for bringing an “inventive antibody” into contact with a sample to thereby form a complex-A of “the peptide comprising the amino acid sequence represented by SEQ ID NO:1—the inventive antibody”.

The explanation of the “sample” is similar to that described above. Also, the explanation of the “inventive antibody” is similar to that described above. The method for bringing the sample into contact with the inventive antibody is not particularly limited, so long as the inventive antibody is brought into contact with the “peptide comprising the amino acid sequence represented by SEQ ID NO:1” in the sample.

The sample is brought into contact with the inventive antibody to thereby form a complex-A of “the peptide comprising the amino acid sequence represented by SEQ ID NO:1—the inventive antibody”. As a result, a “mixture comprising the complex-A and the inventive antibody which does not form the complex-A” is obtained by step (a).

Step (b):

Step (b) is a step for bringing the “mixture comprising the complex-A and the inventive antibody which does not form the complex-A” obtained in step (a) into contact with a “solid phase on which a peptide consisting of the amino acid sequence represented by SEQ ID NO:1 is immobilized” to thereby form a complex of “the peptide consisting of the amino acid sequence represented by SEQ ID NO:1 immobilized on the solid phase—the inventive antibody”.

The explanation of the solid phase to which the “peptide consisting of the amino acid sequence represented by SEQ ID NO:1” is immobilized is similar to that in Inventive method-1.

The explanation of the “peptide consisting of the amino acid sequence represented by SEQ ID NO:1” immobilized on the solid phase is also similar to that described above. Instead of this peptide, a “peptide consisting of the amino acid sequence represented by SEQ ID NO:2” or a “peptide consisting of the amino acid sequence represented by SEQ ID NO:3” can also be used.

Also, the explanation of the method for immobilizing the “peptide consisting of the amino acid sequence represented by SEQ ID NO: I” on the solid phase is similar to that in Inventive method-1, and may use a usual method such as a physical adsorption method and a covalent bond method. Among these, a physical adsorption method is preferred because of its simple operation and wide use.

The contact between the “solid phase on which a peptide consisting of the amino acid sequence represented by SEQ ID NO:1 is immobilized” and the mixture obtained in step (a) can be carried out as described above. Similarly to the above, the solid phase and the liquid phase are separated after the reaction, and it is preferred that the surface of the solid phase is washed with a washing solution, if necessary, to remove any non-specifically adsorbed substances or non-reacted components in the sample. The washing solution which can be used is also similar to that described above.

As a result of bringing the “solid phase on which the peptide consisting of the amino acid sequence represented by SEQ ID NO:1 is immobilized” into contact with the mixture obtained in step (a), the “inventive antibody which does not form the complex-A” is bound to the “peptide consisting of the amino acid sequence represented by SEQ ID NO: I” to thereby form a complex of “the peptide consisting of the amino acid sequence represented by SEQ ID NO:1 immobilized on the solid phase—the inventive antibody”.

Step (c):

Step (c) is a step for detecting the complex formed in step (b). The method for detecting the complex is not particularly limited. When the “inventive antibody” which is labeled with a label or is capable of being labeled with a label is used, the label is detected by a method similar to that in Inventive method-1 to thereby detect the complex. Furthermore, the detection of the complex is preferably carried out by using an “antibody which specifically binds to the inventive antibody and which is labeled with a label or is capable of being labeled with a label”.

Also, the explanations of the “antibody which specifically binds to the inventive antibody”, the label which can be used for labeling, a labeling method, a label detecting method and the like are similar to those in Inventive method-1. However, since this method is an inhibition method, detection of the label in a large amount means that the inventive antibody which does not form the complex-A of “the peptide comprising the amino acid sequence represented by SEQ ID NO:1—the inventive antibody” is present in a large amount (correspondingly, the complex-A is present in a small amount), that is, the “peptide comprising the amino acid sequence represented by SEQ ID NO:1” is present in a small amount in the sample.

<3>-1 Inventive Kit 1

Inventive kit-1 is a kit for assaying a “peptide comprising the amino acid sequence represented by SEQ ID NO:1”, which comprises the following components (A) and (B):
(A) a solid phase; and
(B) an inventive antibody.

The inventive antibody used in Inventive kit-1 is preferably one which is labeled with a label or is capable of being labeled with a label.

The explanations of the “solid phase”, the “inventive antibody”, the “label”, the method for labeling the antibody with a label and the “peptide comprising the amino acid sequence represented by SEQ ID NO:1” as an assaying target in Inventive kit-1 are similar to those in “<2>-Inventive method”. This inventive kit can be used for assaying the “peptide comprising the amino acid sequence represented by SEQ ID NO:1” by means of direct ELISA.

<3>-2 Inventive Kit 2

Inventive kit-2 is a kit for assaying a “peptide comprising the amino acid sequence represented by SEQ ID NO:1”, which comprises the following components (A) and (13):
(A) a solid phase on which an inventive antibody (primary antibody) is immobilized; and
(B) an inventive antibody (secondary antibody).

The “secondary antibody” used in Inventive kit-2 is preferably one which is labeled with a label or is capable of being labeled with a label.

The explanations of the “inventive antibodies (primary antibody, secondary antibody)”, the “solid phase on which an inventive antibody (primary antibody) is immobilized”, the “label”, the method for labeling the antibody with a label, and the “peptide comprising the amino acid sequence represented by SEQ ID NO:1” as an assaying target in Inventive kit-2 are similar to those in “<2>-Inventive method”. This inventive kit can be used for assaying the “peptide comprising the amino acid sequence represented by SEQ ID NO:1” by means of a sandwich method.

<3>-3 Inventive Kit 3

Inventive kit-3 is a kit for assaying a “peptide comprising the amino acid sequence represented by SEQ ID NO:1”, which comprises the following components (A), (B) and (C):
(A) a solid phase on which a peptide consisting of the amino acid sequence represented by SEQ ID NO:1 is immobilized;
(B) an inventive antibody; and
(C) an antibody which specifically binds to the inventive antibody and which is labeled with a label or is capable of being labeled with a label.

The explanations of the “solid phase on which a peptide consisting of the amino acid sequence represented by SEQ ID NO:1 is immobilized”, the “inventive antibody”, the “antibody which specifically binds to the inventive antibody”, the “label”, the method for labeling the antibody with a label and the “peptide comprising the amino acid sequence represented by SEQ ID NO:1” as an assaying target in Inventive kit-3 are similar to those in “<2>-Inventive method”. This inventive kit can be used for assaying a “peptide comprising the amino acid sequence represented by SEQ ID NO:1” by means of an inhibition method.

Assay using Inventive kit-1 can be carried out according to Inventive method-1, assay using Inventive kit-2 can be carried out according to Inventive method-2, and assay using Inventive kit-3 can be carried out according to Inventive method-3.

<4> Inventive Detection Method

The inventive detection method is a method for detecting a bacterial pneumonia wherein an antigen in a sample which can be detected by an “inventive antibody” or an “antibody capable of specifically binding to CAP18” is assayed and, as a result, a bacterial pneumonia in a patient from which the sample is obtained is detected.

The “inventive antibody” and the “antibody capable of specifically binding to CAP18” is preferably one which is labeled with a label or is capable of being labeled with a label.

The explanations of the “inventive antibody”, the “label”, the method for labeling the antibody with a label, the “sample” and the “peptide comprising the amino acid sequence represented by SEQ ID NO:1” as an assaying target in this inventive detection method are similar to those in “<2>-Inventive method”.

CAP18 is preferably mammalian CAP18, and more preferably human CAP18.

The “antibody capable of specifically binding to CAP18” is not particularly limited, so long as it can specifically bind to CAP18, and the binding site is not particularly limited.

The inventive detection method is preferably carried out immunologically by using the “inventive antibody” or the “antibody capable of specifically binding to CAP18”, and examples include “<2>-Inventive method” described above. The “antibody capable of specifically binding to CAP18” can be used, instead of the “inventive antibody”.

In the inventive detection method, the detection of a bacterial pneumonia is preferably carried out by evaluating or monitoring the presence or absence of infection, degree or type of the bacterial pneumonia. Specifically, these detections can be carried out by comparing the assaying results of a sample containing an antigen capable of being detected by the “inventive antibody” or the “antibody capable of specifically binding to CAP18” with the assaying results of a sample which does not contain the antigen.

The detection of CAP18 is not limited to the above-described method, and may be carried out by a usual peptide detection method, such as high performance liquid chromatography

<5> Inventive Diagnostic Kit

The inventive diagnostic kit is a kit for diagnosing a bacterial pneumonia, which comprises an “inventive antibody”.

The “inventive antibody” used in the inventive diagnostic kit is preferably one which is labeled with a label or is capable of being labeled with a label.

The explanations of the “inventive antibody”, the “label” and the method for labeling the antibody with a label in this inventive diagnostic kit is similar to those in “<2>-Inventive method” described above.

The inventive diagnostic kit is preferably a kit of either one of inventive kits 1 to 3, and assay using an Inventive kit-1 can be carried out according to Inventive method-1, assay using an Inventive kit-2 can be carried out according to Inventive method-2, and assay using an Inventive kit-3 can be carried out according to Inventive method-3.

<6> Inventive Diagnostic Agent

The inventive diagnostic agent is an agent for diagnosing a bacterial pneumonia, which comprises an “inventive antibody”.

The “inventive antibody” used in the inventive diagnostic agent is preferably one which is labeled with a label or is capable of being labeled with a label.

The explanations of the “inventive antibody”, the “label” and the method for labeling the antibody with a label in this inventive diagnostic agent are similar to those in “<2>-Inventive method” described above.

The inventive diagnostic agent can contain a pharmaceutically acceptable carrier.

The present invention is described in detail based on Examples.

### EXAMPLE 1

Production of Inventive Antibody:

(1) Production of Monoclonal Antibody

A peptide consisting of the amino acid sequence represented by SEQ ID NO:1 (FR KSKEK IGKEF KRIVQ RIKDF LRNLV; hereinafter referred to as “27 amino acids-peptide”) was synthesized, bound to a hemocyanin (keyhole limpet hemocyanin), and then intraperitoneally administered to a mouse (Balb/c) (purchased from Clea Japan, 8 week-old) 4 times in total at 25 μg as an initial dose and thereafter at 10 μg for immunization (using as an adjuvant a Freund complete adjuvant (manufactured by Wako Pure Chemical) initially and thereafter a Freund incomplete adjuvant (manufactured by Wako Pure Chemical)). Spleen cells were taken from the immunized mouse, and then subjected to a cell fusion with mouse myeloma cells (cell line: P3-X63-Ag8.653 (653: ATCC No. CRL 1588)) using polyethylene glycol 4000 (PEG4000) to thereby prepare a hybridoma.

The resultant hybridoma was proliferated continuously, and a hybridoma cell line which continuously produces an antibody which specifically binds to the 27 amino acids-peptide was selected.

The selected hybridoma cell line was cultured in a hollow fiber cell culture device using a serum-free medium (trade name: CD hybridoma, manufactured by Invitrogen), and the culture supernatant was taken and dialyzed against PBS to obtain a monoclonal antibody (Toyo6E3). The antibody belongs to the subclass IgG1.

(2) Production of Polyclonal Antibody

A gene encoding the 27 amino acids-peptide was cloned into pET17b vector (pET17b-CAP18), then a region encoding the sequence excluding the signal peptides (420 bp of 91-510, 140 amino acids) was then further inserted into pET19b-CAP18 for re-cloning, and then transformation was carried out in E. coli DH5α. The inserted gene sequence was confirmed and then transformed again in E. coli BL21 (DE3) for protein expression, which was induced by IPTG (isopropyl-β-D-thiogalactopyranoside) to express the protein. The protein was subjected to affinity purification using an Ni-NTA (manufactured by Novagene) to obtain recombinant CAP18.

The human-derived recombinant CAP18 (rCAP18) was subcutaneously administered to a rabbit at 0.1 μg for immunization (by using as an adjuvant a Freund complete adjuvant (manufactured by Wako Pure Chemical) initially and thereafter a Freund incomplete adjuvant (manufactured by Wako Pure Chemical)). Two to three weeks after the initial immunization, booster immunization (0.1 to 0.2 mg/rabbit, 4 to 6 times) was carried out by a standard method, and about 1 week after the final immunization, the blood was taken and the serum was separated. The serum was heated to inactivate complements, and then subjected to 33% saturated ammonium sulfate precipitation to thereby obtain a polyclonal antibody.

### EXAMPLE 2

Immunostaining of Cell by Monoclonal Antibody Toyo6E3:

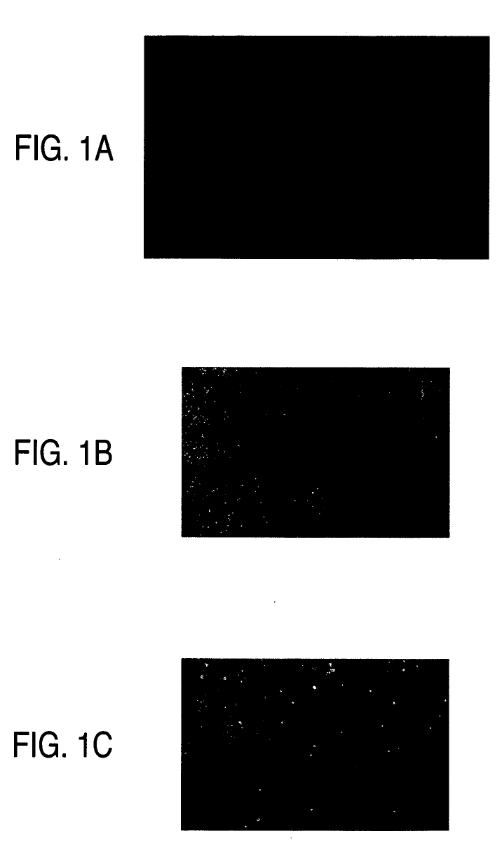

Using the monoclonal antibody (Toyo6E3) produced in Example 1(1) as a primary antibody and a goat anti-mouse IgG antibody (manufactured by Jackson) labeled with an Alexa Fluor® 488 (manufactured by Molecular Probes) as a secondary antibody, human peripheral neutrophiles were stained according to a usual method. The fluoromicroscopic image (magnification: 1000) is shown in FIG. 1A.

The immune electromicroscopic images of human peripheral neutrophiles using Toyo6E3 as a primary antibody and a goat anti-mouse IgG antibody (manufactured by Jackson) labeled with a 20 nm gold particle (manufactured by British Bioicell) as a secondary antibody are shown in FIGS. 1B (magnification: 5000) and 1C (magnification: 10000).

FIG. 1 shows that the monoclonal antibody Toyo6E3 can be used in cell staining.

Also, FIG. 1A shows that a region which was considered to be the cytoplasm of the neutrophile can be stained by Toyo6E3. Furthermore, FIGS. 1B and C show that granulocytes of the neutrophile can be stained by Toyo6E3.

### EXAMPLE 3

Detection and Determination of Peptide by Direct ELISA:

The 27 amino acids-peptide and a “peptide consisting of the amino acid sequence represented by SEQ ID NO:2” (KEF KRIVQ RIKDF LRNLV; hereinafter referred to as “18 amino acids-peptide”) were each dissolved in a carbonate buffer (pH 9.6) at various concentrations, and these solutions were added to wells of a polystyrene microtiter plate. Thereafter, the plate was stored at about 22° C. for 1 hour to physically adsorb the peptide to the plate. Subsequently, the solution was removed and the surface of the plate was washed with a phosphate buffer (pH 7.5) supplemented with 0.05% Tween 20. Each peptide immobilized on this plate was assayed by the method (i) or (ii) described below.
(i) A method in which, after reacting the monoclonal antibody produced in Example 1(1) (Toyo6E3) as a primary antibody and a goat anti-mouse IgG antibody (manufactured by Jackson) labeled with a horse radish peroxidase (HRP) as a secondary antibody, a TMB chromogenic substrate (manufactured by Nippon Bio-Rad) was used to develop a color, and an absorption at 450 nm was measured to thereby assay the peptide immobilized on the solid phase.
(ii) A method in which, after reacting the polyclonal antibody produced in Example 1(2) as a primary antibody and a goat anti-rabbit IgG antibody labeled with the HRP as a secondary antibody, a TMB chromogenic substrate was used to develop a color, and an absorption at 450 nm (OD450) was measured to thereby assay the peptide immobilized on the solid phase.

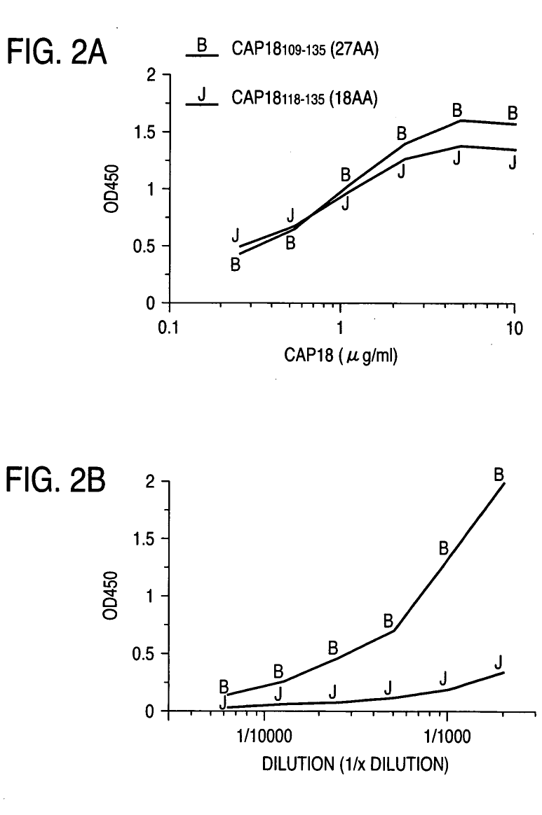

The results of method (i) are shown in FIG. 2A, and the results of method (ii) are shown in FIG. 2B. The curve indicated by “B” in the drawings corresponds to the plate on which the 27 amino acids-peptide was immobilized, and the curve indicated by “J” in the drawings corresponds to the plate on which the 18 amino acids-peptide was immobilized.

FIG. 2A shows that both of the 27 amino acids-peptide and the 18 amino acids-peptide can be detected and determined by the direct ELISA using the “monoclonal antibody Toyo6E3”. Also, since Toyo6E3 shows a reactivity to the 18 amino acids-peptide at a degree similar to that to the 27 amino acids-peptide, it was suggested that it specifically reacts with the 18 amino acids-peptide.

Also, FIG. 2B shows that both of the 27 amino acids-peptide and the 18 amino acids-peptide can be detected and determined by the direct ELISA using the “polyclonal antibody produced in Example 1(2)”. However, because of its low reactivity to the 18 amino acids-peptide, this polyclonal antibody was indicated to be suitable for the detection and determination of the 27 amino acids-peptide.

Furthermore, these results suggest that this polyclonal antibody mainly recognizes the region which is contained in the 27 amino acids-peptide but not contained in the 18 amino acids-peptide (SEQ ID NO:3; FRKSKEKIG).

### EXAMPLE 4

Detection and Determination of CAP18 by Indirect ELISA (Sandwich Method):

The polyclonal antibody produced in Example 1(2) was dissolved in a phosphate buffer (pH 7.4), and the solution was added to wells of a polystyrene microtiter plate. Thereafter, the plate was stored at about 22° C. for 1 hour to physically adsorb the peptide to the plate. Subsequently, the surface of the plate was washed with a phosphate buffer (pH 7.4) supplemented with 0.05% Tween 20, and then blocked with a phosphate buffer supplemented with 1% BSA (bovine serum albumin). To this plate, the peptide and recombinant CAP18 (rCAP18; derived from human) were added at various concentrations, followed by incubation at 22° C. for 3 hours. After the incubation, the plate was washed with a phosphate buffer.

Then, the monoclonal antibody (Toyo6E3) produced in Example 1(1) (secondary antibody) was added to the plate, followed by incubation at 22° C. for 2 hours. After the incubation, the plate was washed with a phosphate buffer.

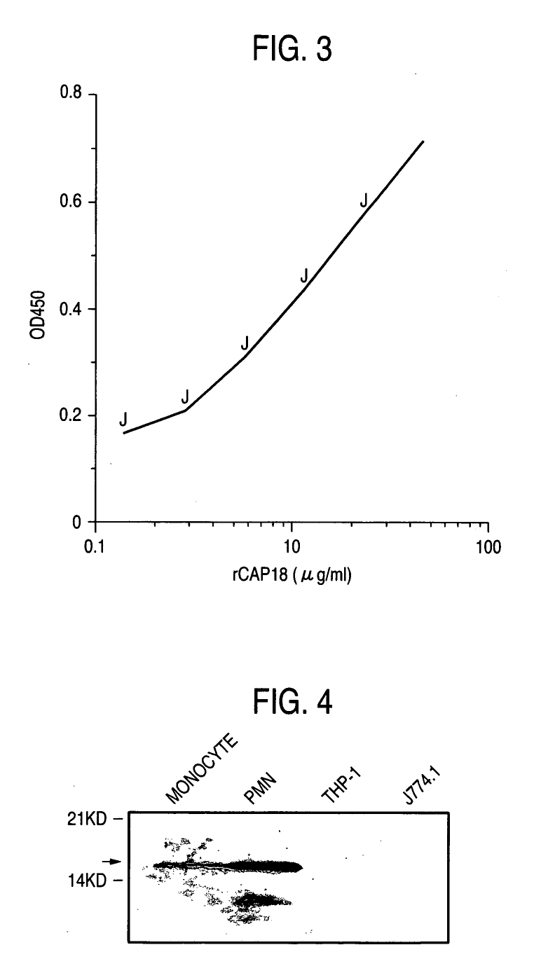

Then, a goat anti-mouse IgG antibody labeled with the HRP was allowed to react similarly, a TMB chromogenic substrate was used to develop a color, and an absorption at 450 nm (OD450) was measured to thereby determine the peptide or rCAP18. The results are shown in FIG. 3.

FIG. 3 shows that CAP18 can also be detected and determined by the indirect ELISA (sandwich method) using the “monoclonal antibody Toyo6E3” or the “polyclonal antibody produced in Example 1(2)”.

### EXAMPLE 5

Detection of CAP18 in Cell Extract by western blotting:

Proteins in cell extracts of human peripheral blood mononuclear cells (monocytes), human peripheral neutrophiles (PMN), a human monocyte-derived cell line (THP-1) and a mouse macrophage-like cell line (J774.1) were separated by SDS-polyacrylamide gel electrophoresis, and electrically transferred onto a nitrocellulose membrane. This membrane was blocked with a 3% skimmed milk, and then allowed to react with the monoclonal antibody (Toyo6E3) produced in Example 1(1). Then, the goat anti-mouse IgG antibody labeled with HRP was added as a secondary antibody, and allowed to react similarly, and then the band developed by ECL was detected. The results are shown in FIG. 4.

FIG. 4 shows that CAP18 can also be detected and determined by Western blotting using the “monoclonal antibody Toyo6E3”.

It was also suggested that, in the human peripheral neutrophile fraction, 18 kDa-size CAP18 is present in a large amount and a lower molecular antibody-binding protein (considered to be formed by enzymatic cleavage of the lipopolysaccharide-binding region (LPS-binding region) of the CAP18 molecule) is also present in a small amount. A small amount of the 18 kDa CAP18 was also detected in the human peripheral monocyte fraction, whereas no substance which binds to Toyo6E3 was detected in the human monocyte-derived THP-1 or the mouse macrophage-like cell line J774.1.

### EXAMPLE 6

Detection of CAP18 in Serum by Immunoprecipitation:

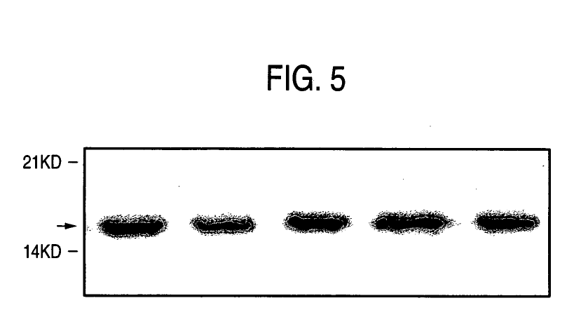

Using the monoclonal antibody produced in Example 1(1) (Toyo6E3), CAP18 contained in the serum of healthy persons (n=5) was immunoprecipitated. The immunoprecipitated material was detected by Western blotting using the same monoclonal antibody. The results are shown in FIG. 5. In this connection, the procedure of Western blotting was similar to that in Example 5.

FIG. 5 shows that the “monoclonal antibody Toyo6E3” can be also used in the immunoprecipitation and that CAP18 can be detected by the immunoprecipitation using this antibody.

### EXAMPLE 7

Release of CAP18 from Human Neutrophiles Stimulated by fMLP:

Human peripheral neutrophiles (PN) or mononuclear cells (monocytes) (2×10⁶/well/200 ml) were incubated in the presence of 0 nM, 1 nM or 10 nM formylmethionylleucylphenylalanine (fMLP) at 37° C. for 90 minutes. Thereafter, each of a culture supernatant fraction (150 ml) and a cell-rich fraction (50 ml) was recovered, and in the latter fraction, cells were lysed to prepare an extract.

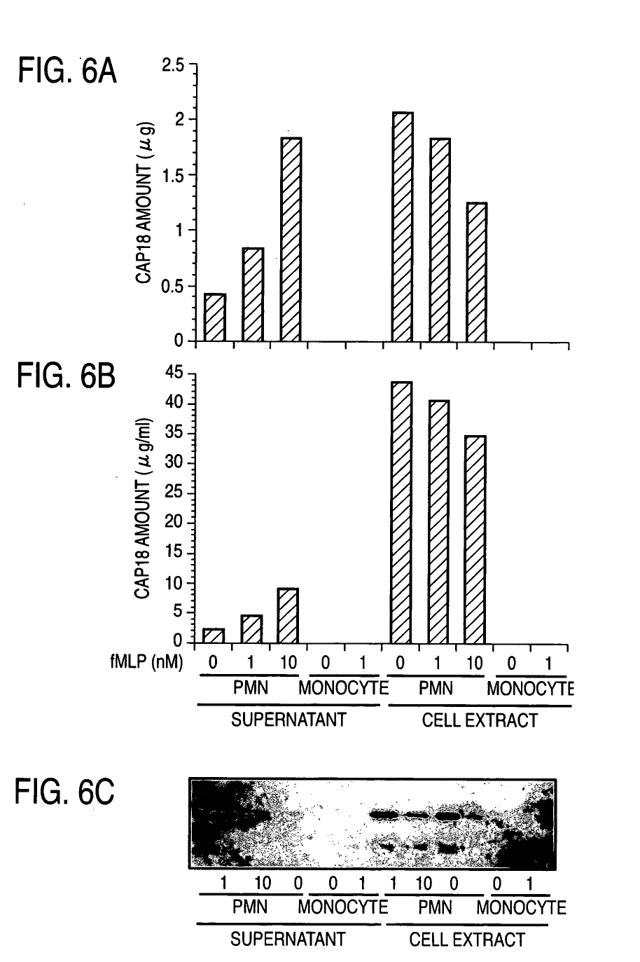

The amount of CAP18 contained in each of the supernatant fractions and cell extracts was assayed by the indirect ELISA using the monoclonal antibody (Toyo6E3) produced in Example 1(1). The results are shown in FIG. 6B. Also, based on the data in FIG. 6B, the “amount of the released CAP18” and the “amount of the intracellular CAP18” were estimated and the results are shown in FIG. 6A. The fraction recovered in FIG. 6B was also detected by Western blotting using the monoclonal antibody (Toyo6E3), and the results are shown in FIG. 6C. The procedure of Western blotting was similar to that in Example 5, and the cell-rich fraction was used after 3-fold dilution.

It was found from FIG. 6C that CAP18 is released into a culture supernatant in response to the fMLP stimulation and that the release depends on the fMLP stimulation (concentration) (FIGS. 6A and B), by the ELISA using the monoclonal antibody Toyo6E3. The size of the released CAP18 was 18 kDa, and no fragment of a small molecular weights was detected (FIG. 6C).

### EXAMPLE 8

The indirect ELISA (sandwich method) described in Example 4 was used to assay CAP18 in sputum of each of serious pneumonia patients having different causative bacteria. The results are shown below.

| | Assayed value |
|---|---|
| Subject to be assayed | of CAP18 |
| Patient A (disease: Klebsiela pneumonia) | 1.486 μg/ml |
| Patient B (disease: methicillin-resistant | 1.682 μg/ml |
| Staphylococcus aureus) | |

In any patients, the CAP18 concentration in the sputum was markedly increased in comparison with comparative examples (control) shown below.

As comparative examples, the CAP18 concentration present in sputum of each of patients who exhibited no particular inflammatory sings and were bedridden for a long period and in sputum of a healthy human were assayed by the indirect ELISA (sandwich method) as described above. The results are shown below.

| | Assayed value |
|---|---|
| Subject to be assayed | of CAP18 |
| Patient C (Pseudomonas aeruginosa colonization, | Not detected |
| No fever, No increase of leukocytes) | |
| Patient D (Pseudomonas aeruginosa and Staphylococcus | 0.674 μg/ml |
| colonization, No fever, No increased leukocyte) | |
| Healthy person E (smoker, healthy) | |
| Sample 1 | Not detected |
| Sample 2 | Not detected |

In any patients of the comparative examples, CAP18 was not detected at all or was detected only at a slightly increased level. No CAP18 was detected in the healthy person.

Based on the results described above, CAP18 in sputum can serve as an index which gives a marked reflection of human neutrophile-derived alveolar and interstitial inflammatory signs, and early diagnosis and monitoring of a bacterial pneumonia can be carried out by assaying CAP18 in a sample such as sputum.

### EXAMPLE 9

Production of Inventive Kit:

(1) Inventive kit-1 having the following constitution was produced.
1. 96-Well immunoplate 1
2. Monoclonal antibody (Toyo6E3) produced in Example 1(1) 1 (primary antibody)
3. HRP-Labeled goat anti-mouse IgG antibody 1 (secondary antibody)
4. TMB Solution 1
5. Aqueous hydrogen peroxide 1
6. Reaction-stopping solution (1M HCl) 1
(2) Inventive kit-2 having the following constitution was produced.
1. 96-Well immunoplate on which the polyclonal antibody 1 (Toyo6E3) produced in Example 1(2) is immobilized
2. Monoclonal antibody (Toyo6E3) produced in Example 1(1) 1 (primary antibody)
3. HRP-Labeled goat anti-mouse IgG antibody 1 (secondary antibody)
4. TMB Solution 1
5. Aqueous hydrogen peroxide 1
6. Reaction-stopping solution (1M HCl) 1
(3) Inventive kit-2 having the following constitution was produced.
1. 96-Well immunoplate on which the 27 amino acids 1-peptide is immobilized
2. Monoclonal antibody (Toyo6E3) produced in Example 1(1) 1
3. HRP-Labeled goat anti-mouse IgG antibody 1 (secondary antibody)
4. TMB Solution 1
5. Aqueous hydrogen peroxide 1
6. Reaction-stopping solution (1M HCl) 1

While the present invention has been described in detail and with reference to specific embodiments thereof, it will be apparent to one of skill in the art that various changes and modifications can be made therein without departing from the spirit and scope thereof.

This application is based on Japanese application Nos. 2002-2213040 and 2003-70932 filed on Jul. 22, 2002 and Mar. 14, 2003, respectively, the entire contents of which are incorporated hereinto by reference. All references cited herein are incorporated in their entirety.

### INDUSTRIAL APPLICABILITY

The inventive antibody is an antibody which specifically binds to a peptide characteristic of CAP18, and is very useful because it can be used as a tool for detecting or assaying CAP18. The inventive antibody is highly homogeneous and reproducible, and can also permanently be produced in a large amount, resulting advantageously in remarkable reduction in production costs. Furthermore, the inventive antibody is very useful since it can be used in the inventive method or in production of the inventive kit.

The inventive method is extremely useful since it can be applied to a research reagent or a diagnostic agent for diseases relating to CAP18 and the like and can also be possibly to applied to monitoring of the diseases and the like. For example, it can be used in a method for evaluating respiratory diseases such as chronic pulmonary disease, chronic airway disease, acute pulmonary disease, inflammatory pulmonary disease, adult respiratory distress syndrome (ARDS), bacterial pneumonia, interstitial pneumonia and upper airway bronchitis, which comprises assaying a CAP18 amount in a living sample and relating the assayed result to the diseases.

Furthermore, the inventive kit is extremely useful since the inventive method can carried out more rapidly and more conveniently by using the inventive kit.

Free Text of Sequence Listing

SEQ ID NO:1—explanation of artificial sequence: synthetic peptide
SEQ ID NO:2—explanation of artificial sequence: synthetic peptide
SEQ ID NO:3—explanation of artificial sequence: synthetic peptide

1. An antibody which binds to a “peptide consisting of the amino acid sequence represented by SEQ ID NO:1”.

2. The antibody according to claim 1, which specifically binds to a “peptide consisting of the amino acid sequence represented by SEQ ID NO:2”.

3. The antibody according to claim 1, which is a monoclonal antibody.

4. The antibody according to claim 1, which belongs to the immunoglobulin subclass IgG1.

5. The antibody according to claim 1, which specifically binds to a “peptide consisting of the amino acid sequence represented by SEQ ID NO:3”.

6. The antibody according to claim 1, which is a polyclonal antibody.

7. A method for assaying a “peptide comprising the amino acid sequence represented by SEQ ID NO:1” in a sample, which comprises using an antibody described in of claim 1.

8. The method according to claim 7, wherein said assaying of the “peptide comprising the amino acid sequence represented by SEQ ID NO:1” is carried out by steps comprising the following steps (a) and (b):
(a) bringing a solid phase into contact with a sample to thereby immobilize the “peptide comprising the amino acid sequence represented by SEQ ID NO:1” in the sample on the solid phase; and
(b) detecting the “peptide comprising the amino acid sequence represented by SEQ ID NO:1” immobilized on the solid phase in step (a) by using an “antibody described in claim 1”.

9. The method according to claim 8, wherein the “antibody described in claim 1” is labeled with a label or is capable of being labeled with a label.

10. The method according to claim 8, wherein said detecting of the “peptide comprising the amino acid sequence represented by SEQ ID NO:1” immobilized on the solid phase is carried out by further using an “antibody which specifically binds to the antibody described in of claim 1 and which is labeled with a label or is capable of being labeled with a label”.

11. The method according to claim 7, wherein said assaying of the “peptide comprising the amino acid sequence represented by SEQ ID NO:1” is carried out by steps comprising the following steps (a) and (b):
(a) bringing a “solid phase on which an antibody described in claim 1 as a primary antibody is immobilized”, a “sample”, and an “antibody described in claim 1 as a secondary antibody” into contact to thereby form a sandwich complex of “the primary antibody immobilized on the solid phase—the peptide comprising the amino acid sequence represented by SEQ ID NO:1—the secondary antibody”; and
(b) detecting the sandwich complex formed in step (a).

12. The method according to claim 11, wherein said assaying of the “peptide comprising the amino acid sequence represented by SEQ ID NO:1” is carried out by steps comprising the following steps (a) to (c):
(a) bringing a sample into contact with a “solid phase on which an antibody described in claim 1 as a primary antibody is immobilized” to thereby form a complex of “the primary antibody immobilized on the solid phase—the peptide comprising the amino acid sequence represented by SEQ ID NO:1”;
(b) bringing an “antibody described in claim 1 as a secondary antibody” into contact with the solid phase to thereby form a sandwich complex of “the primary antibody immobilized on the solid phase—the peptide comprising the amino acid sequence represented by SEQ ID NO:1—the secondary antibody”; and
(c) detecting the sandwich complex formed in step (b).

13. The method according to claim 11, wherein the secondary antibody is labeled with a label or is capable of being labeled with a label.

14. The method according to claim 11, wherein said detecting of the complex is carried out by using an “antibody which specifically binds to the secondary antibody and which is labeled with a label or is capable of being labeled with a label”.

15. The method according to claim 7, wherein said assaying of the “peptide comprising the amino acid sequence represented by SEQ ID NO:1” is carried out by steps comprising the following steps (a) and (b):
(a) bringing a “solid phase on which a peptide consisting of the amino acid sequence represented by SEQ ID NO:1 is immobilized”, a “sample”, and an “antibody described in claim 1” into contact to thereby form a complex of “the peptide consisting of the amino acid sequence represented by SEQ ID NO:1 immobilized on the solid phase—the antibody described in claim 1” and a complex of “the peptide comprising the amino acid sequence represented by SEQ ID NO:1 in the sample—the antibody described in claim 1”; and
(b) detecting at least one of the complex of “the peptide consisting of the amino acid sequence represented by SEQ ID NO:1 immobilized on the solid phase—the antibody described in claim 1” and the complex of “the peptide comprising the amino acid sequence represented by SEQ ID NO:1 in the sample—the antibody according to claim 1”.

16. The method according to claim 15, wherein said assaying of the “peptide comprising the amino acid sequence represented by SEQ ID NO:1” is carried out by steps comprising the following steps (a) to (c):
(a) bringing a sample into contact with an “antibody described in claim 1” to thereby form a complex-A of “the peptide comprising the amino acid sequence represented by SEQ ID NO:1—the antibody described in claim 1”;
(b) bringing a “mixture comprising the complex-A and the antibody described in claim 1 which does not form the complex-A” obtained in step (a) into contact with a “solid phase on which the peptide consisting of the amino acid sequence represented by SEQ ID NO:1 is immobilized” to thereby form a complex of “the peptide consisting of the amino acid sequence represented by SEQ ID NO:1 immobilized on the solid phase—the antibody described in claim 1”; and
(c) detecting the complex formed in step (b).

17. The method according to claim 15, wherein the “antibody described in claim 1” is labeled with a label or is capable of being labeled with a label.

18. The method according to claim 15, wherein said detecting of the complex is carried out by using an “antibody which specifically binds to the antibody according to claim 1 and which is labeled with a label or is capable of being labeled with a label”.

19. The method according to claim 7, wherein the sample is a body fluid.

20. A kit for assaying a “peptide comprising the amino acid sequence represented by SEQ ID NO:1”, which comprises the following components (A) and (B):
(A) a solid phase; and
(B) an antibody described in claim 1.

21. The kit according to claim 20, wherein the “antibody described in claim 1” is labeled with a label or is capable of being labeled with a label.

22. A kit for assaying a “peptide comprising the amino acid sequence represented by SEQ ID NO:1”, which comprises the following components (A) and (B):
(A) a solid phase on which an antibody described in claim 1 as a primary antibody is immobilized; and
(B) an antibody described in claim 1 as a secondary antibody.

23. The kit according to claim 22, wherein the secondary antibody is labeled with a label or is capable of being labeled with a label.

24. A kit for assaying a “peptide comprising the amino acid sequence represented by SEQ ID NO:1”, which comprises the following components (A), (B) and (C):
(A) a solid phase on which a peptide consisting of the amino acid sequence represented by SEQ ID NO:1 is immobilized;
(B) an antibody described in claim 1; and
(C) an antibody which specifically binds to the antibody described in claim 1 and which is labeled with a label or is capable of being labeled with a label.

25. A method for detecting a bacterial pneumonia, which comprises assaying an antigen in a sample which can be detected by an “antibody described in claim 1” or an “antibody capable of specifically binding to CAP18” to thereby detect a bacterial pneumonia in a patient from which the sample is obtained.

26. The method according to claim 25, wherein the antigen in the sample is selected from the group consisting of a “peptide comprising the amino acid sequence represented by SEQ ID NO:1”, a “peptide comprising the amino acid sequence represented by SEQ ID NO:2”, a “peptide comprising the amino acid sequence represented by SEQ ID NO:3”, and CAP18.

27. The method according claim 25, wherein said assaying is immunologically carried out by using an antibody selected from the group consisting of an “antibody capable of binding to a peptide consisting of the amino acid sequence represented by SEQ ID NO:1”, an “antibody capable of specifically binding to a peptide consisting of the amino acid sequence represented by SEQ ID NO:2”, an “antibody capable of specifically binding to a peptide consisting of the amino acid sequence represented by SEQ ID NO:3”, and an “antibody capable of specifically binding to CAP18”.

28. The method according to claim 25, wherein said detecting of a bacterial pneumonia is carried out by evaluating or monitoring the presence or absence of infection, degree or type of the bacterial pneumonia.

29. The method according to claim 25, wherein said assaying is carried out by a method described in claim 7.

30. A kit for diagnosing a bacterial pneumonia, which comprises an antibody described in claim 1.

31. The kit according to claim 30, which consists of any one of a kit described in claim 20.

32. A diagnostic agent which comprises, as an active ingredient, an antibody according to claim 1.
